(19) United States
(12) Patent Application Publication
(10) Pub. No.: US 2018/0188219 A1
(43) Pub. Date: Jul. 5, 2018

(54) GAS SENSOR AND METHOD OF MANUFACTURE THEREOF

(71) Applicants: Rohm and Haas Electronic Materials LLC, Marlborough, MA (US); Rohm and Haas Electronic Materials Korea Ltd., Cheonan-si (KR); Dow Global Technologies LLC, Midland, MI (US)

(72) Inventors: Eui Hyun Ryu, Yongin (KR); Peter Trefonas, III, Medway, MA (US); Bok-Hee Lee, Osan (KR); Phillip D. Hustad, Midland, MI (US)

(21) Appl. No.: 15/846,640

(22) Filed: Dec. 19, 2017

Related U.S. Application Data
(60) Provisional application No. 62/440,432, filed on Dec. 30, 2016.

(51) Int. Cl. G01N 33/00; G01N 27/12; G01N 5/02

(52) U.S. Cl. CPC G01N 33/0047; G01N 5/02; G01N 27/126

(57) ABSTRACT

Disclosed herein is a gas sensor comprising a piezoelectric substrate; and a first polymeric layer disposed on the substrate; where the first polymeric layer has a first surface contacting a substrate and a second surface having a higher surface area than the first surface, where the first polymeric layer comprises a repeat unit that is effective to adsorb molecules present in the atmosphere.

### CROSS REFERENCE TO RELATED APPLICATIONS

This application is a Non-Provisional application which claims the benefit of U.S. Provisional Application No. 62/440,432, filed Dec. 30, 2016, which is incorporated by reference in its entirety herein.

### BACKGROUND

This disclosure relates to a gas sensor and to a method of manufacture thereof.

Gas sensors are used for detecting the presence of hazardous, unwanted and disagreeable gases in the home as well as in industry. Examples of such gases are carbon monoxide, carbon dioxide, formaldehyde, hydrogen sulfide, amines, ozone, ammonia, benzene, and so on. In order to detect these hazardous gases, functionalized polymers that have display weak interactions such as hydrogen bonding, van der Waals interactions, π-π interaction and electrostatic interactions with the gases are used. These functionalized polymers are generally coated on the surface of sensor electrodes, which are in direct contact with piezo-electric sensors used in the chemical sensor.

During this detection process, a weight change from the captured hazardous, unwanted and disagreeable gases are transformed to an electric current by a piezo-electric process. Therefore, the sensitivity of the gas sensor is dependent on the amount of gas that contacts the detection surface of the sensor. In order to improve detection capabilities, it is therefore desirable to increase the surface area of the contact surface of the sensor.

### SUMMARY

Disclosed herein is a gas sensor comprising a piezoelectric substrate; and a first polymeric layer disposed on the substrate; where the first polymeric layer has a first surface contacting a substrate and a second surface having a higher surface area than the first surface, where the first polymeric layer comprises a repeat unit that is effective to adsorb molecules present in the atmosphere.

Disclosed herein too is a method of manufacturing a gas sensor comprising disposing upon a piezoelectric substrate a first polymeric layer having a first surface that contacts the substrate and a second surface that has a higher surface area than the first surface; and where the first polymeric layer comprises repeat units that are operative to undergo hydrogen bonding, van der Waals interactions, π-π interactions, electrostatic interactions, or a combination thereof with an ambient gaseous molecule.

Disclosed herein is a method of detecting a gas comprising contacting a gas sensor with a gaseous molecule; where the gas sensor comprises a piezoelectric substrate; and a first polymeric layer having a first surface that contacts the substrate and a second surface that has a higher surface area than the first surface; forming at least one of a hydrogen bond, a van der Waals interaction, a π-π interaction or an electrostatic interaction between the gas molecule and the first polymeric layer; and determining an identity of the gas molecule based on a difference of the sensor prior to and after the forming of the hydrogen bond, the van der Waals interaction, the π-π interaction and/or the electrostatic interaction.

### BRIEF DESCRIPTION OF THE FIGURES

FIG. 1 depicts an exemplary exploded view of a gas sensing device assembly;

FIG. 2(A) depicts one exemplary embodiment of the substrate with the first and second polymeric layers disposed thereon;

FIG. 2(B) depicts another exemplary embodiment of the substrate with the first and second polymeric layers disposed thereon;

FIG. 3 depicts molecules having different solubility parameters;

FIG. 4 depicts another method of manufacturing the sensing element; and

FIG. 5 depicts another method of manufacturing the sensing element.

### DETAILED DESCRIPTION

As used herein, “phase-separate” refers to the propensity of the blocks of block copolymers to form discrete microphase-separated domains, also referred to as “microdomains” or “nanodomains” and also simply as “domains”. The blocks of the same monomer aggregate to form periodic domains, and the spacing and morphology of domains depends on the interaction, size, and volume fraction among different blocks in the block copolymer. Domains of block copolymers can form during application, such as during a spin-casting step, during a heating step, or can be tuned by an annealing step. “Heating”, also referred to herein as “baking”, is a general process wherein the temperature of the substrate and coated layers thereon is raised above ambient temperature. “Annealing” can include thermal annealing, thermal gradient annealing, solvent vapor annealing, or other annealing methods. Thermal annealing, sometimes referred to as “thermal curing” can be a specific baking process for fixing patterns and removing defects in the layer of the block copolymer assembly, and generally involves heating at elevated temperature (e.g., 150° C. to 400° C.), for a prolonged period of time (e.g., several minutes to several days) at or near the end of the film-forming process. Annealing, when performed, is used to reduce or remove defects in the layer (referred to as a “film” hereinafter) of microphase-separated domains.

The self-assembling layer comprising a block copolymer having at least a first block and a second block that forms domains through phase separation. “Domain”, as used herein, means a compact crystalline, semi-crystalline, or amorphous region formed by corresponding blocks of the block copolymer, where these regions may be lamellar, cylindrical, spherical, or form a bicontinuous network and are formed orthogonal or perpendicular to the plane of the surface of the substrate and/or to the plane of a surface modification layer disposed on the substrate, or alternatively formed parallel or in plane with the substrate. In an embodiment, the domains may have an average largest dimension of about 2 to about 75 nanometers (nm), specifically about 4 to about 50 nm, and still more specifically about 7 to about 30 nm.

The term “M_{N}” used herein and in the appended claims in reference to a block copolymer of the present invention is the number average molecular weight of the block copolymer (in g/mol) determined according to the method used herein in the Examples.

The term “M_{W}” used herein and in the appended claims in reference to a block copolymer of the present invention is the weight average molecular weight of the block copolymer (in g/mol) determined according to the method used herein in the Examples.

The term “PDI” or “D” used herein and in the appended claims in reference to a block copolymer of the present invention is the polydispersity (also called polydispersity index or simply “dispersity”) of the block copolymer determined according to the following equation:

$$PDI = \frac{{M}_{W}}{{M}_{N}} .$$

Disclosed herein is a gas sensor and a sensor element for the gas sensor that improves the sensitivity of detection of undesirable hazardous gases in the atmosphere. The sensor element comprises a substrate on which is disposed a first polymeric layer. In one embodiment, the substrate is a piezoelectric substrate (crystal) that converts a sensor element weight change into an electrical signal. In another embodiment, the sensing element facilitates the detection of gas molecules by virtue of a change of electrical current that travels through the polymeric layer. The first polymeric layer has a first surface that contacts the substrate and a second surface that is opposed to the first surface which has a higher surface area than the first surface. In an exemplary embodiment, the second surface has a textured surface. The texture increases the surface area significantly over the same surface when it is not textured. The second surface is exposed to the atmosphere.

In an embodiment, the first polymeric layer comprises a copolymer that phase segregates into separate phases. One of the phases is removed (e.g., etched), leaving behind a first polymeric layer that has a textured surface. After the removal of one of the phases of the copolymer, a second polymeric layer may optionally be disposed on the textured first polymeric layer. The first polymeric layer may undergo phase separation into lamellar, cylindrical, spherical, and ordered or disordered bicontinuous (also known as fingerprint) morphologies. In an embodiment, the gas sensor comprises only a single polymeric layer—the textured first polymeric layer.

When a gas molecule contacts a free surface (where the free surface is a surface that contacts the ambient atmosphere) of the first polymeric or the optional second polymeric layer, it interacts with the free surface by hydrogen bonding, van der Waals interactions, π-π interactions, electrostatic interactions, or a combination thereof, which increases the weight of the sensor element. The piezoelectric crystal converts the weight difference of the sensing element to an electrical signal, which is then used to determine the identity of the hazardous gas.

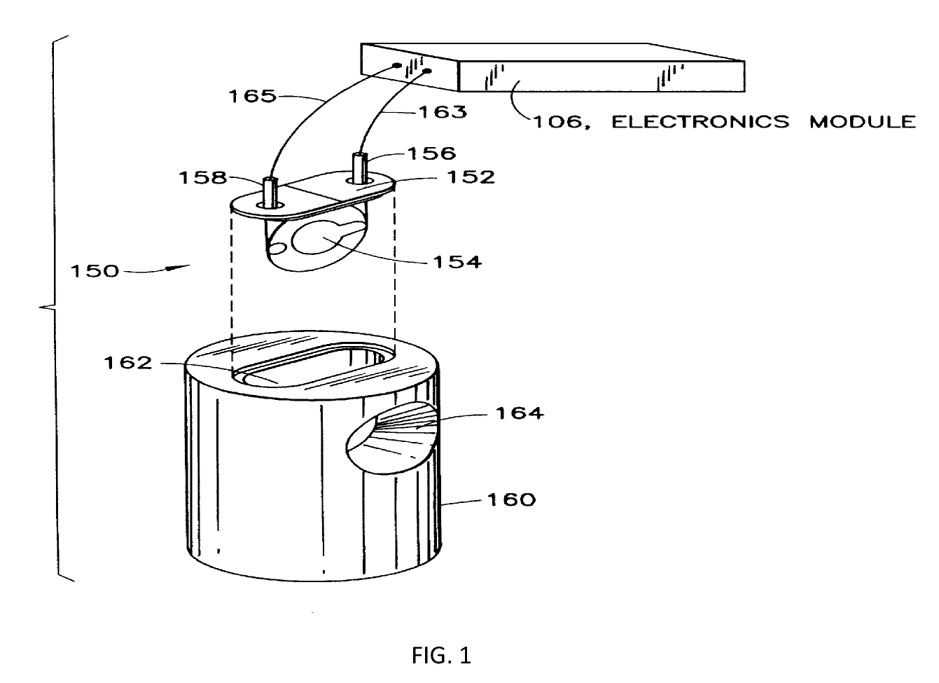

The FIG. 1 shows an exploded view of a gas sensing device assembly 100 that comprises a sensor element 150 and a housing 160. The sensor element 150 comprises a substrate 154 which is coated with the first polymeric layer and the optional second polymeric layer 155 (hereinafter multilayered coating 155) that interacts with a fluid component of interest to yield an interaction product of differing mass characteristic than the original multilayered coating. In an embodiment, the substrate 154 comprises a piezoelectric crystal.

The substrate 154 with the multilayered coating disposed thereon (also referred to herein as a coated substrate) is mounted on the plug member 152, with the respective leads of the substrate 154 protruding exteriorly of the plug member when the plug member is engaged with the housing 160 with the coated substrate extending into the cavity 162. The housing 160 features an opening 164 by which a gas can flow into the cavity 162 containing the sensor element 150. Although not shown in the front perspective view of the FIG. 1, the housing 160 has another opening therein, opposite opening 164 and in register with such opening, for discharge from the housing of the fluid component that flows past the sensor element 150.

The leads 156 and 158 of the sensor element 150 may contact suitable electronics as shown schematically as electronics module 166 in the FIG. 1, by which the presence a concentration of the hazardous gas species can be detected. The electronics module 166 contacts the sensor element leads 156 and 158 by wires 163 and 165, respectively.

Electronics module 106 provides the functions of (i) sampling the output resonant frequency of the piezoelectric crystal while an oscillating electric field is applied thereto, (ii) determining the change in resonant frequency from the fundamental resonant frequency incident to the formation of the interaction product when the sensor material interacts with the gas species in the fluid being monitored, and (iii) generating an output indicative of the presence of the gas species in such fluid.

In a specific embodiment of the sensor assembly shown in the FIG. 1, the housing 160 may comprise a metal or plastic housing which has the cavity 162 machined into it for the insertion of the sensor element 150, as well as two feedthrough openings (opening 164 and the opposite opening not shown in FIG. 1) for the gas being monitored to flow through the sensor. In the body of this housing is the flow restricting orifice. Front end driver electronics are plugged directly onto the legs (leads 156 and 158) of the sensor assembly.

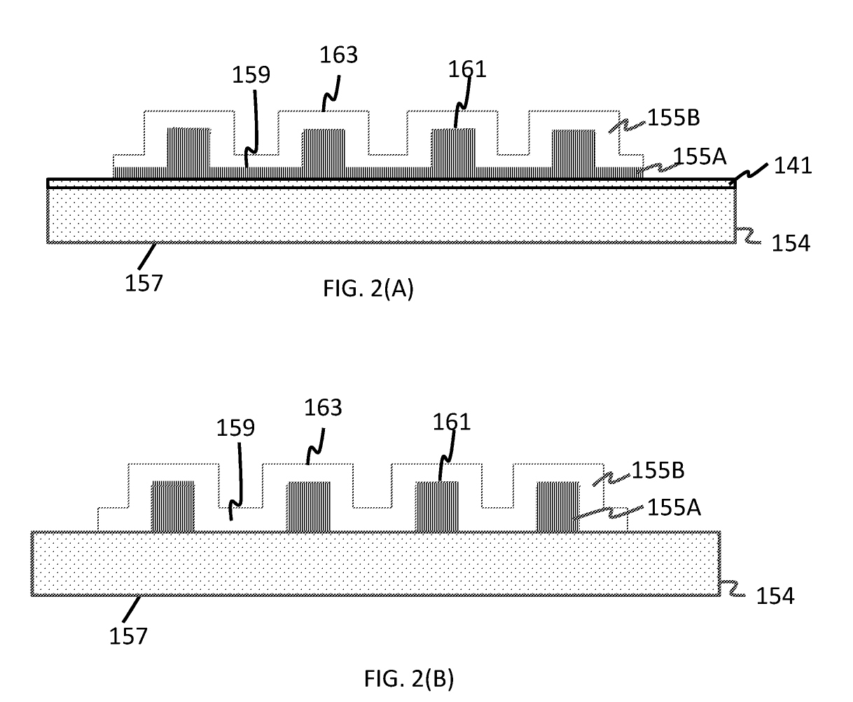

The FIGS. 2(A) and 2(B) show the substrate 154 with the multilayered coating 155 disposed thereon. Suitable substrates 154 are those that display piezoelectric properties. Examples of such substrates are quartz, berlinite (AlPO₄), topaz, tourmaline-group minerals, lead titanate (PbTiO₃), langasite (La₃Ga₅SiO₁₄), a quartz-analogous crystal; gallium orthophosphate (GaPO₄), also a quartz-analogous crystal; lithium niobate (LiNbO₃), lithium tantalate (LiTaO₃), barium titanate (BaTiO₃), lead zirconate titanate (Pb[ZrₓTi₁₋ₓ]O₃ with 0≤x≤1)—more commonly known as PZT, potassium niobate (KNbO₃), sodium tungstate (Na₂WO₃), Ba₂NaNb₅O₅, Pb₂KNb₅O₁₅, zinc oxide (ZnO), polyvinylidene fluoride (PVDF), or the like, or a combination thereof.

The multilayered coating 155 comprises the first polymeric layer 155A and the optional second polymeric layer 155B. The first polymeric layer 155A has opposing surfaces 157 and 159, where surface 157 (hereinafter the first surface 157) contacts the substrate 154. An optional surface modification layer 141 may be disposed on the substrate 154. As will be detailed later, the surface modification layer is optional and comprises a random copolymer that is covalently bonded to the substrate 154 to form a brush polymer. The second surface 159 is textured and contacts the optional second polymeric layer 155B. The textured second surface 159 may be a zig-zag texture, a square wave texture, a sinusoidal texture, or the like, or a combination thereof.

In an embodiment, the optional second polymeric layer 155B has a first surface 161 and a second surface 163. The first surface 161 of the second polymeric layer 155B contacts the second surface 159 of the first polymeric layer 155A. In an embodiment, the second surface 163 of the second polymeric layer 155B is parallel to the second surface 159 of the second polymeric layer 155B.

The first polymeric layer 155A is a copolymer that comprises at least two different repeat units where each repeat unit is a part of a polymer. The copolymer thus comprises two or more different polymers. In an embodiment, the copolymer comprises a first polymer and a second polymer. A chi parameter that measures interactions between a first polymer and the second polymer is 0.003 to 0.15 at a temperature of 200° C. The copolymer may be a block copolymer (e.g., a diblock copolymer or a triblock copolymer), an alternating copolymer, a random copolymer, a gradient copolymer, a graft copolymer, a star block copolymer, an ionomer, a bottlebrush block copolymer or a combination comprising at least one of the foregoing polymers. In an exemplary embodiment, the copolymer is a block copolymer.

When the copolymer is a block copolymer having a first polymer (that comprises the first repeat unit) and a second polymer (that comprises the second repeat unit), the chi parameter that measures interactions between the first polymer and the second polymer is 0.003 to 0.15 at a temperature of 200° C.

In other words, the copolymer that forms the first layer 155A comprises a first polymer and a second polymer that are chemically dissimilar and that are characterized by an energetic penalty of dissolving when one polymer into the other polymer. In block copolymers, this energetic penalty applies to the dissolving of one block polymer into the other block polymer. This energetic penalty is characterized by the Flory-Huggins interaction parameter or “chi” (denoted by χ) and is an important factor in determining microphase segregation behavior in block copolymers. Accordingly, the χ value of a block copolymer defines a tendency of the block copolymer to segregate into microdomains as a function of the block copolymer's weight, chain length, and/or degree of polymerization. The chi parameter can often be approximated from the square of the difference in Hildebrand solubility parameters of the respective polymers of the copolymer. In an exemplary embodiment, the chi parameter has a value of 0.003 to 0.15 at a temperature of 200° C.

As used herein, the χ parameter denotes the segment-segment interaction parameter associated with a segment volume of 0.118 cubic nanometers (nm³). The molecular weight of a segment, Mo, in units of g/mol is equal to the segment volume multiplied by the polymer density and divided by Avogadro's number. Also as used herein, the degree of polymerization, N, is defined as the number of segments per block copolymer molecule and MN=N×Mo.

A greater chi parameter between the first block of the copolymer with respect to the second block of the copolymer promotes the formation of smaller, highly periodic lamellar and/or cylindrical domains, which can be used to produce periodic structures in the substrate upon which the copolymer is disposed.

The chi parameter χ₁₂ may also be represented by the equation:

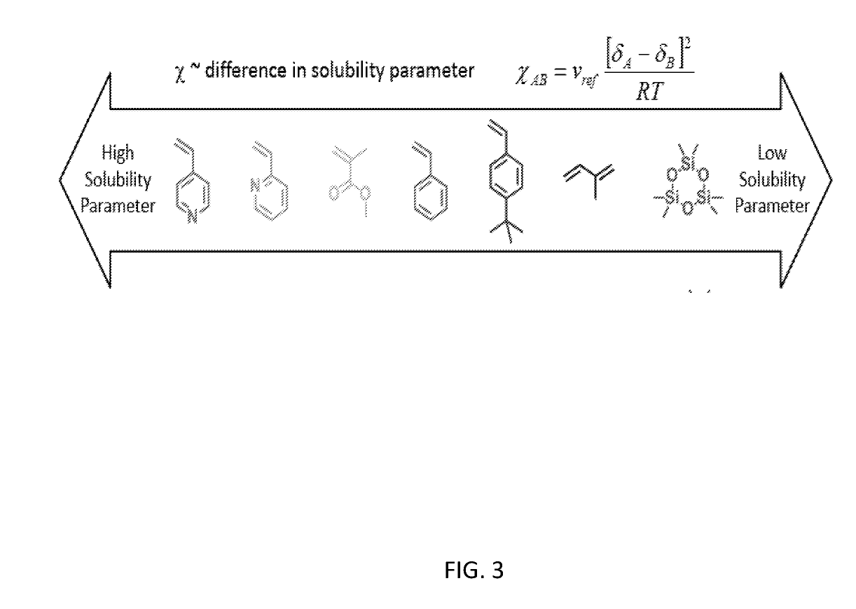

χ₁₂=V_{seg}(δₐ−δ_{b})²/RT

where χ₁₂ represents the chi parameter, Vseg is the segment volume, δₐ and δ_{b} are the solubility parameters of the first polymer and the second polymer respectively, R is the gas constant and T is the temperature. For a block copolymer, δₐ and δ_{b} are the solubility parameters of the first polymer and the second polymer respectively. The FIG. 3 is an exemplary depiction of the variation in the solubility parameters of different polymers. Polyvinylpyridines (such as poly(4-vinylpyridine) and poly(2-vinylpyridine) have relatively high solubility parameters, while dialkylsiloxane polymers have relatively low solubility parameters.

In an embodiment, the first polymer and the second polymer may be different polymers (where the chi parameter has a value of 0.0010 to 0.150 at a temperature of 200° C.) selected from the group consisting of a polyacetal, a polyacrylic, a polycarbonate, a polystyrene, a polyester, a polyamide, a polyamideimide, a polyarylate, a polyarylsulfone, a polyethersulfone, a polyphenylene sulfide, a polyvinyl chloride, a polysulfone, a polyimide, a polyetherimide, a polytetrafluoroethylene, a polyetherketone, a polyether etherketone, a polyether ketone ketone, a polybenzoxazole, a polyoxadiazole, a polybenzothiazinophenothiazine, a polybenzothiazole, a polypyrazinoquinoxaline, a polypyromellitimide, a polyquinoxaline, a polybenzimidazole, a polyoxindole, a polyoxoisoindoline, a polydioxoisoindoline, a polytriazine, a polypyridazine, a polypiperazine, a polypyridine, a polypiperidine, a polytriazole, a polypyrazole, a polypyrrolidine, a polycarborane, a polyoxabicyclononane, a polydibenzofuran, a polyphthalide, a polyanhydride, a polyvinyl ether, a polyvinyl thioether, a polyvinyl alcohol, a polyvinyl ketone, a polyvinyl halide, a polyvinyl nitrile, a polyvinyl ester, a polysulfonate, a polynorbornene, a polysulfide, a polythioester, a polysulfonamide, a polyurea, a polyphosphazene, a polysilazane, a polyurethane, or the like, or a combination including at least one of the foregoing polymers.

In an embodiment, the first or the second block (but not both the first and the second block) may comprise a polymer derived from an acrylate or acrylic acid monomer having a structure represented by formula (1):

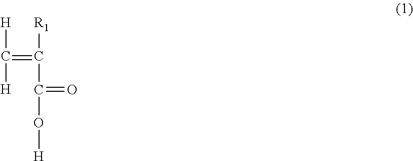

where R₁ is a hydrogen or an alkyl group having 1 to 10 carbon atoms. Examples of the first repeat monomer are acrylates and alkyl acrylates such as, for example, methyl acrylates, ethyl acrylates, propyl acrylates, acrylic acid, or the like, or a combination comprising at least one of the foregoing acrylates.

In one embodiment, the first or the second block (but not both the first and the second block) may comprise a polymer derived from an acrylate monomer having a structure represented by formula (2):

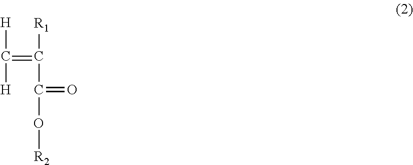

where R₁ is a hydrogen or an alkyl group having 1 to 10 carbon atoms and R₂ is a C₁₋₁₀ alkyl, a C₃₋₁₀ cycloalkyl, or a C₇₋₁₀ aralkyl group. Examples of the (meth)acrylates are methacrylate, ethacrylate, propyl acrylate, methyl methacrylate, methyl ethylacrylate, methyl propylacrylate, ethyl ethylacrylate, methyl arylacrylate, or the like, or a combination comprising at least one of the foregoing acrylates. The term “(meth)acrylate” implies that either an acrylate or methacrylate is contemplated unless otherwise specified.

As noted above, the first or the second block (but not both the first and the second block) may comprise a polymer derived from an acrylate monomer having a structure represented by formula (3):

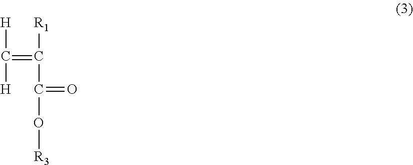

where R₁ is a hydrogen or an alkyl group having 1 to 10 carbon atoms and R₃ is a C₂₋₁₀ fluoroalkyl group. Examples of compounds having the structure of formula (3) are trifluoroethyl methacrylate, and dodecafluoroheptylmethacrylate.

In another embodiment, the first or the second block (but not both the first and the second block) may be a polymer derived from a vinyl aromatic monomer. The vinyl aromatic monomer of the second block is preferably of the following general formula (4):

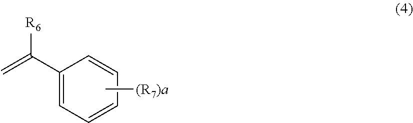

wherein: R₆ is chosen from hydrogen and C₁ to C₃ alkyl or haloalkyl such as fluoro-, chloro-, iodo- or bromoalkyl, with hydrogen being typical; R₇ is independently chosen from hydrogen, halogen (F, Cl, I or Br), and optionally substituted alkyl such as optionally substituted C₁ to C₁₀ linear or branched alkyl or C₃ to C₈ cyclic alkyl, optionally substituted aryl such as C₅ to C₂₅, C₅ to C₁₅ or C₅ to C₁₀ aryl or C₆ to C₃₀, C₆ to C₂₀ or C₆ to C₁₅ aralkyl, and optionally including one or more linking moiety chosen from —O—, —S—, —C(O)O— and —OC(O)—, wherein two or more R₂ groups optionally form one or more rings, for example, fused rings such as naphthyl, anthracenyl and the like; and a is an integer from 0 to 5.

Suitable vinyl aromatic monomers of the formula (4) include monomers chosen, for example, from the following:

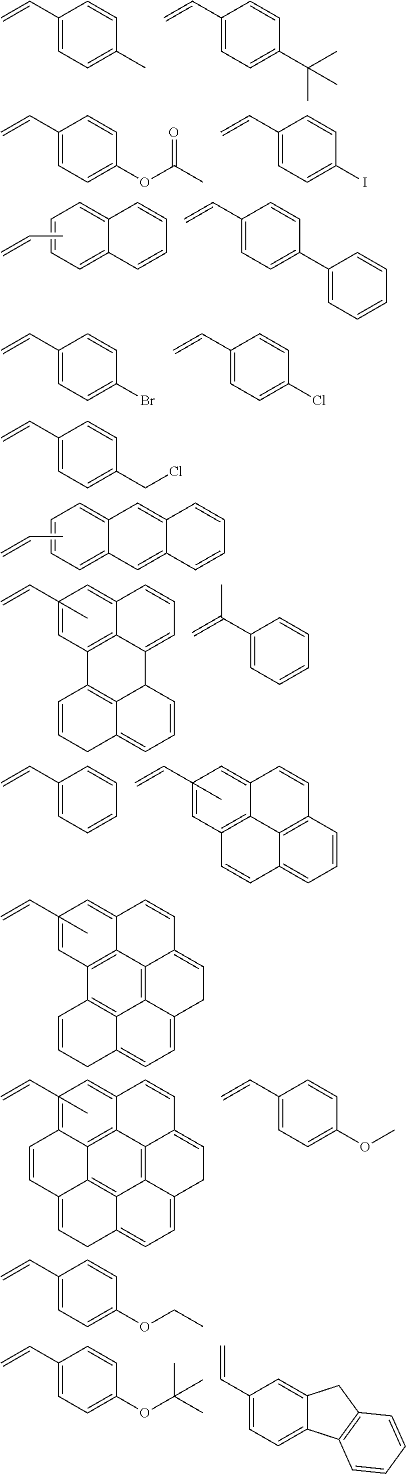

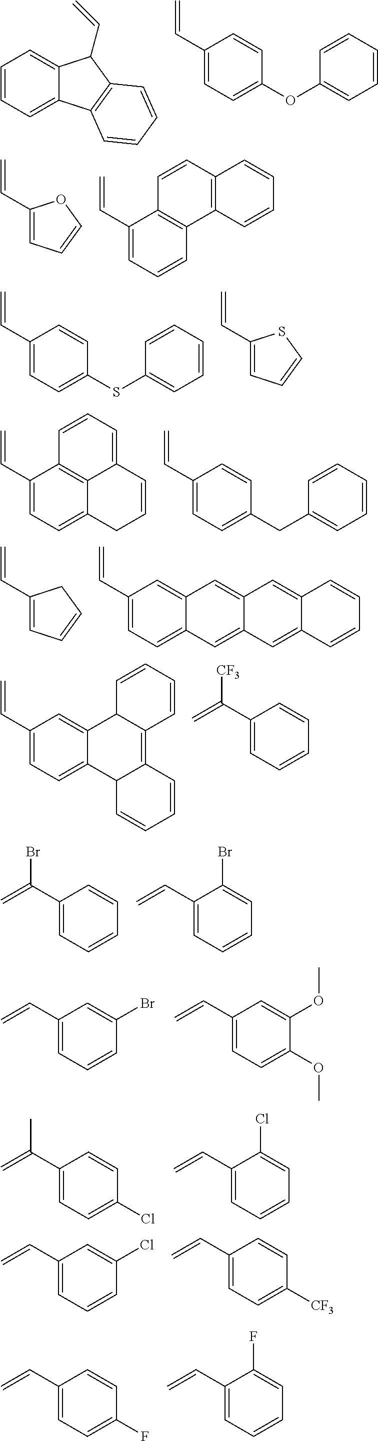

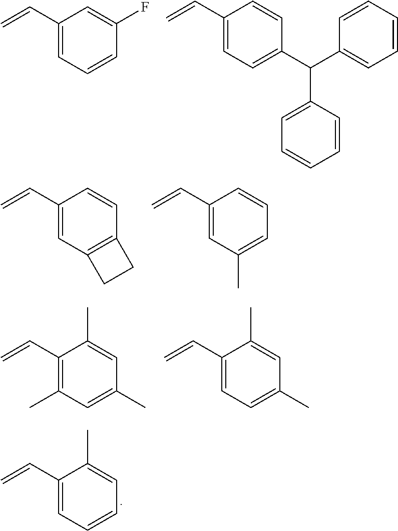

Examples of suitable vinyl aromatic monomers are styrene, o-methylstyrene, p-methylstyrene, m-methylstyrene, α-methylstyrene, o-ethylstyrene, m-ethylstyrene, p-ethylstyrene, a-methyl-p-methylstyrene, 2,4-dimethylstyrene, monochlorostyrene, p-tert-butylstyrene, 4-tert-butylstyrene, or the like, or a combination comprising at least one of the foregoing vinyl aromatic monomers. Exemplary vinyl aromatic monomers for use in either the first block or the second block are styrene and 4-tert-butylstyrene.

In another embodiment, the first or the second block (but not both the first and the second block) may be a polymer derived from a siloxane monomer. Polysiloxanes derived from a siloxane monomer and having a repeating unit with the structure of formula (5)

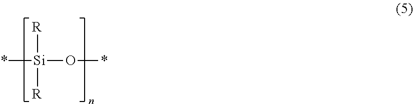

wherein each R is independently a C₁-C₁₀ alkyl, a C₃-C₁₀ cycloalkyl, a C₆-C₁₄ aryl, a C₇-C₁₃ alkylaryl or a C₇-C₁₃ arylalkyl and where n is 10 to 10,000. Combinations of the foregoing R groups can be present in the same monomer. Exemplary siloxanes include dimethylsiloxane, diethylsiloxane, diphenylsiloxanes, and combinations thereof.

In yet another embodiment, the first and second polymer may be derived from a monomer that comprises a nitrogen-containing group. Examples of nitrogen-containing groups include, for example, amine groups and amide groups, for example, primary amines such as amine, secondary amines such as alkylamines including N-methylamine, N-ethylamine, N-t-butylamine, and the like, tertiary amines such as N,N-dialkylamines including N,N-dimethylamine, N,N-methylethylamine, N,N-diethylamine, and the like. Useful amide groups include alkylamides such as N-methylamide, N-ethylamide, N-phenylamide, N,N-dimethylamide, and the like. The nitrogen-containing groups can also be part of a ring, such as pyridine, indole, imidazole, triazine, pyrrolidine, azacyclopropane, azacyclobutane, piperidine, pyrrole, purine, diazetidine, dithiazine, azocane, azonane, quinoline, carbazole, acridine, indazole, benzimidazole, and the like. Preferred nitrogen containing groups are amine groups, amide groups, pyridine groups, or a combination thereof.

In an embodiment, the first or second polymer may comprise a nitrogen containing group as shown below in the Formulas (6) to (11),

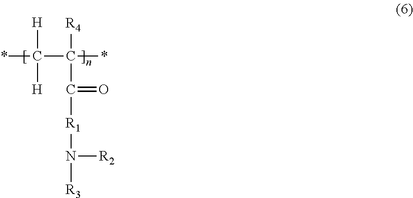

where n is the number of repeat units, and where R₁ is a C₁ to C₃₀ alkyl group, preferably a C₂ to C₁₀ alkyl group, R₂ and R₃ can be the same or different and can be hydrogen, a hydroxyl, a C₁ to C₃₀ alkyl group, preferably a C₁ to C₁₀ group, and wherein R₄ is a hydrogen or a C₁ to C₃₀ alkyl group,

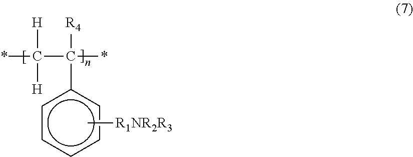

where n, R₁, R₂, R₃ and R₄ are defined above in the Formula (6).

A preferred form of the structure of the Formula (7) is shown below in the Formula (8):

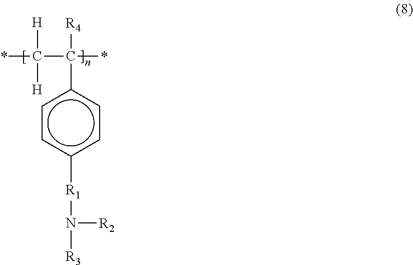

where the R₁NR₂R₃ group is located at the para-position, and where n, R₁, R₂, R₃ and R₄ are defined above in the Formula (6).

Another example of a hydrogen acceptor containing block that comprises a nitrogen containing group is shown below in the Formula (9)

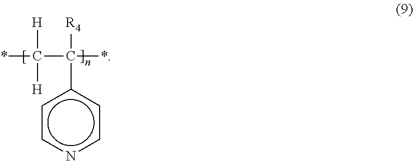

In the formula (9), n and R₄ are defined in Formula (6) and the nitrogen atom can be in the ortho, meta, para positions or any combination thereof (e.g., in both the ortho and para positions).

Yet another example of a hydrogen acceptor containing block that comprises a nitrogen containing group are shown below in the Formula (10)

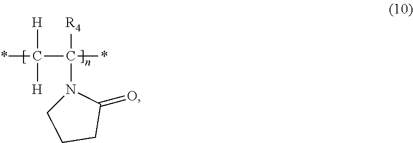

where n and R₄ are defined above in the Formula (6).

Yet another example of a hydrogen acceptor containing block that comprises a nitrogen containing group are poly(alkylene imines) shown below in the Formula (11)

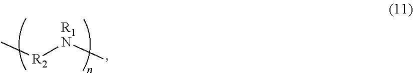

where R₁ is a 5 membered ring that is substituted with 1-4 nitrogen atoms, R₂ is a C₁ to C₁₅ alkylene and n represents the total number of repeat units. An example of the structure of Formula (11) is polyethyleneimine. Exemplary structures of the hydrogen acceptor of the Formula (11) are shown below.

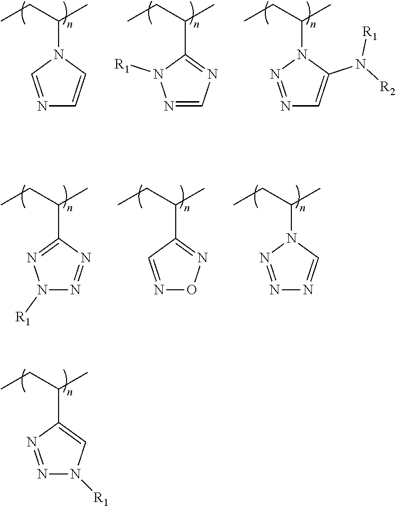

Exemplary block copolymers that are contemplated for use in the first layer 155A include diblock or triblock copolymers such as poly(styrene-b-vinyl pyridine), poly(styrene-b-butadiene), poly(styrene-b-isoprene), poly(styrene-b-methyl methacrylate), poly(styrene-b-alkenyl aromatics), poly(isoprene-b-ethylene oxide), poly(styrene-b-(ethylene-propylene)), poly(ethylene oxide-b-caprolactone), poly(butadiene-b-ethylene oxide), poly(styrene-b-t-butyl (meth)acrylate), poly(methyl methacrylate-b-t-butyl methacrylate), poly(ethylene oxide-b-propylene oxide), poly(styrene-b-tetrahydrofuran), poly(styrene-b-isoprene-b-ethylene oxide), poly(styrene-b-dimethylsiloxane), poly(styrene-b-trimethylsilylmethyl methacrylate), poly(methyl methacrylate-b-dimethylsiloxane), poly(methyl methacrylate-b-trimethylsilylmethyl methacrylate), poly(methylmethacrylate-b-vinyl pyridine), or the like, or a combination comprising at least one of the foregoing block copolymers.

In an embodiment, the weight average molecular weight of the first polymer is 1,000 to 250,000 grams per mole, while the weight average molecular weight of the second polymer is 1,000 to 250,000 grams per mole. In another embodiment, the first polymer is present in the block copolymer in an amount of 5 to 95 weight percent (wt %), based on the total weight of the copolymer, while the the second polymer is present in the block copolymer in an amount of 5 to 95 weight percent (wt %), based on the total weight of the copolymer.

In an embodiment, the first polymeric layer 155A is manufactured by mixing the copolymer (comprising the first polymer and the second polymer) with a suitable solvent to form a solution and disposing the solution on the substrate.

Suitable solvents that may be used include, for example: alkyl esters such as n-butyl acetate, n-butyl propionate, n-pentyl propionate, n-hexyl propionate and n-heptyl propionate, and alkyl butyrates such as n-butyl butyrate, isobutyl butyrate and isobutyl isobutyrate; ketones such as 2-heptanone, 2,6-dimethyl-4-heptanone and 2,5-dimethyl-4-hexanone; aliphatic hydrocarbons such as n-heptane, n-nonane, n-octane, n-decane, 2-methylheptane, 3-methylheptane, 3,3-dimethylhexane and 2,3,4-trimethylpentane, and fluorinated aliphatic hydrocarbons such as perfluoroheptane; and alcohols such as straight, branched or cyclic C₄-C₉ monohydric alcohol such as 1-butanol, 2-butanol, 3-methyl-1-butanol, isobutyl alcohol, tert-butyl alcohol, 1-pentanol, 2-pentanol, 1-hexanol, 1-heptanol, 1-octanol, 2-hexanol, 2-heptanol, 2-octanol, 3-hexanol, 3-heptanol, 3-octanol and 4-octanol; 2,2,3,3,4,4-hexafluoro-1-butanol, 2,2,3,3,4,4,5,5-octafluoro-1-pentanol and 2,2,3,3,4,4,5,5,6,6-decafluoro-1-hexanol, and C₅-C₉ fluorinated diols such as 2,2,3,3,4,4-hexafluoro-1,5-pentanediol, 2,2,3,3,4,4,5,5-octafluoro-1,6-hexanediol and 2,2,3,3,4,4,5,5,6,6,7,7-dodecafluoro-1,8-octanediol; toluene, anisole and mixtures containing one or more of these solvents. Of these organic solvents, alkyl propionates, alkyl butyrates and ketones, preferably branched ketones, are preferred and, more preferably, C₈-C₉ alkyl propionates, C₈-C₉ alkyl propionates, C₈-C₉ ketones, and mixtures containing one or more of these solvents. Suitable mixed solvents include, for example, mixtures of an alkyl ketone and an alkyl propionate such as the alkyl ketones and alkyl propionates described above. The solvent component is typically present in an amount of from 75 to 99 wt % based on the total weight of the block copolymer and solvent.

As shown above, the FIGS. 2(A) and 2(B), the substrate may be coated with a surface modification layer if desired. The surface modification layer is optional and may comprise a random copolymer that is reacted to the substrate to function as a brush polymer. In an embodiment, the random copolymer may have as its constituents the same polymers as those used in the block copolymer. In another embodiment, the random copolymer may have as its constituents polymers that are different from those used in the block copolymer but which are chemically compatible with those used in the block copolymer.

When a surface modification layer is disposed on the substrate, it may be disposed by spin coating, spray drying, dip coating, and the like. The surface modification layer may be reacted to the surface of the substrate to form brushes or alternatively, the surface modification layer can be cured using either thermal energy and/or electromagnetic radiation. Ultraviolet radiation can be used to cure the surface modification layer. Activators and initiators can be used to vary the curing characteristics of the surface modification film.

The surface modification layer acts like a tying layer interposed between the surface of the substrate and the block copolymer to enhance the adhesion between the block copolymer composition and the substrate.

The surface modification layer may also act to adjust the surface energy to facilitate the formation of desirable segregation morphology in the block copolymer following an annealing step. For example, if the surface modification layer provides a surface energy approximately intermediate between that of the first block and the second block of a block copolymer, this can help to facilitate the self assembly of the block copolymer into vertically arranged domains of each respective block; for example for the case of a block copolymer which naturally forms lamellae when annealed to an equilibrium state, this could provide vertically oriented lamellar domains, or in another example, for the case of a block copolymer which naturally forms cylinders when annealed to an equilibrium state, this could provide vertically oriented cylindrical domains.

The solution of the block copolymer and solvent that enables the formation of the first layer is then disposed on the surface modification layer. The substrate may then be annealed at an elevated temperature of 60 to 250° C. for a period of 10 minutes to 5 hours to facilitate phase separation of the first polymer from the second polymer and to facilitate removal of the solvent.

The first polymer may phase separate from the second polymer to form spheres, cylinders, lamellae, ordered or disordered bicontinuous structures. For gas sensor applications, any of the foregoing forms of phase separation are adequate for forming the sensing surface.

After the phase separation occurs, one of the phases of the block copolymer is optionally removed from the first polymeric layer leaving behind a layer with a textured upper surface on the substrate. The first polymeric layer is therefore the residue of the first block copolymer, where one of the blocks has been etched away. In another embodiment, the first polymeric layer is the residue of a polymer, where a portion of it has been etched away to create the textured surface. In other words, one block of the block copolymer may be removed by etching to produce a textured second surface for the first polymeric layer of the block copolymer. The texturing increases the surface area of the block copolymer. Gas molecules in the atmosphere contact the second surface of the first polymeric layer where they may undergo hydrogen bonding, van der Waals interactions, π-π interactions, electrostatic interactions, or a combination thereof with the repeat units of the first polymeric layer.

In another embodiment, the second block (polymer) of the block copolymer is not removed by etching but is left in place. Gas molecules may diffuse through the second block to contact the second surface of the first polymeric layer where it may undergo hydrogen bonding, van der Waals interactions, π-π interactions, electrostatic interactions, or a combination thereof with the repeat units of the first polymeric layer.

In an embodiment, a first polymeric layer disposed on the substrate has a first surface and a second surface. The first surface contacts the substrate and where the second surface is opposed to the first surface and has a higher surface area than the first surface. When the first polymeric layer comprises a block copolymer, the second surface corresponds to the surface of the first block of the block copolymer. In an embodiment, the entire second surface contacts the ambient atmosphere. In another embodiment, a portion of the second surface contacts the ambient atmosphere, while a portion of the second surface contacts the second block of the block copolymer.

In an embodiment, the entire first surface of the first polymeric layer directly contacts the substrate. In yet another embodiment, the entire first surface of the first polymeric layer directly contacts a surface modification layer that is disposed directly on the substrate. In another embodiment, a portion of the first surface having an area greater than 200 nm×200 nm, preferably greater than 400 nm×400 nm directly contacts a) the substrate or b) an untextured surface modification layer that is disposed on and directly contacts the substrate. In a preferred embodiment, the first polymeric layer comprises molecules of a single polymer.

In an embodiment, a portion of a second polymeric layer is disposed on the first polymeric layer; where the second polymeric layer is derived from a repeat unit that comprises a hydrogen acceptor.

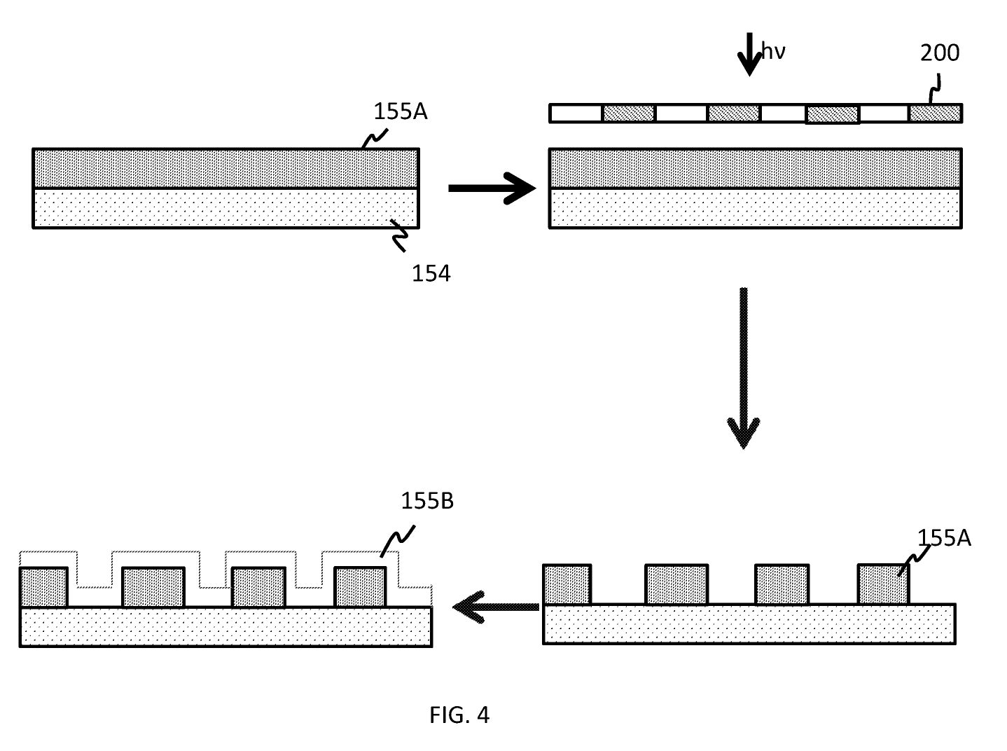

The texturing of the block copolymer is depicted in the FIG. 4, where a photoresist 200 is used to facilitate the etching of one p of the first layer 155A. The layer that is not etched away and left behind on the surface is capable of bonding with certain gaseous molecules in the ambient atmosphere via hydrogen bonding, van der Waals interactions, π-π interactions, electrostatic interactions, or a combination thereof. Examples of such gases are carbon monoxide, carbon dioxide, formaldehyde, hydrogen sulfide, amines, ozone, ammonia, benzene, and so on.

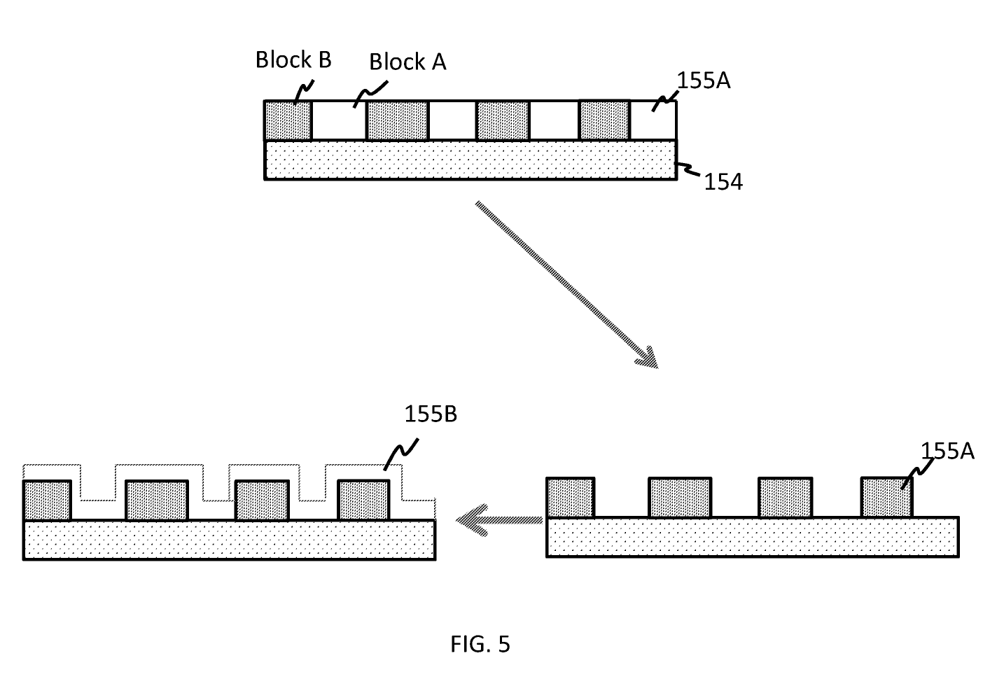

If the polymer used in the first polymeric layer 155A is a copolymer, the first polymer and the second polymer may phase separate (into phase A produced by Block A and phase B produced by Block B) upon being disposed on the substrate 154. This is shown in the FIG. 5. One of the phases of the block copolymer may be etched to form the textured second surface upon which the second polymeric layer 155B is disposed. The first and the second polymeric layers may be subjected to baking if desired. Suitable baking temperatures are from 75 to 200° C., preferably 100 to 150° C.

With reference now to the FIGS. 2(A), 2(B) and 4, in one manner of manufacturing the gas sensor, the first polymeric layer 155A is disposed on the substrate 154. As detailed above, a surface modification layer 141 may be used if desired. The first polymeric layer 155A is part of a first composition that may contain in addition to the copolymer, a solvent. The first composition is first disposed on the substrate 154. The first composition may then be dried by evaporating the solvent to form the first polymeric layer 155A having the first surface and the second surface. A photoresist may then be disposed on second surface of the first polymeric layer. Portions of the first polymeric layer 155A may then be etched to increase the surface area of the second surface. The second surface therefore has a textured surface that is at least twice the surface area of the first surface, preferably at least four times the surface of the first surface. This is depicted in the FIG. 4, where the first polymeric layer 155A is disposed on the surface of the substrate 154. The first polymeric layer may be disposed on the substrate using spin coating, spray painting, dip coating, doctor blading, or the like.

A photoresist 200 is then disposed on the second surface of the first polymeric layer 155A and portions of the first layer 155A are removed using radiation (hv), chemical etching, ion beam etching, or the like, to form a textured second surface. As seen in the FIG. 2(A), only a portion of the first polymeric layer 155A may be removed such that when the second polymeric layer 155B is disposed on the first polymeric layer 155A it contacts a surface of the first polymeric layer 155A along its entire area.

In an embodiment, a second polymeric layer 155B may be disposed on the first polymeric layer 155A after the first polymeric layer 155A is etched. The portions of the first polymeric layer 155A that remain on the substrate after etching are capable of undergoing hydrogen bonding, van der Waals interactions, π-π interactions, electrostatic interactions, or a combination thereof with the polymer used in the second polymeric layer 155B. The second polymeric layer 155B is also capable of bonding with certain gaseous molecules in the ambient atmosphere via hydrogen bonding, van der Waals interactions, π-π interactions, electrostatic interactions, or a combination thereof.

Because the second polymeric layer 155B has a interaction with both the first polymeric layer 155A and certain gaseous molecules, the second polymeric layer 155B can be a homopolymer, a random copolymer, or a block copolymer.

Exemplary polymers used in the second polymeric layer 155B is a homopolyer, a random copolymer, of a block copolymer of poly(4-vinylpyridine), poly(2-vinylpyridine), poly(iso-butyrene), poly(methyl methacylic acid), or the like, or a combination thereof.

After portions of the first polymeric layer 155A are removed, the second polymeric layer 155B is then disposed on the second surface of the first polymeric layer 155A using spin coating, spray painting, dip coating, doctor blading, or the like.

The second polymeric layer 155B may be obtained by disposing on the first polymeric layer 155A a second composition that comprises a solvent as well as a polymer that contains a hydrogen acceptor or a hydrogen donor. If the second polymeric layer 155B contains a protected hydrogen acceptor or a hydrogen donor, it may be deprotected using electromagnetic radiation, thermal decomposition, a photoacid generator, an acid generator, or the like, or a combination thereof. As may be seen in the FIG. 4, the free surface of the second polymeric layer 155B has a higher surface area than the first surface (which is the surface that contacts the substrate) of the first polymeric layer 155A. The free surface of the second polymeric layer 155B is also textured.

As seen in the FIG. 2(B) portions of the first polymeric layer 155A may be removed such that when the second polymeric layer 155B is disposed on the first polymeric layer 155A it contacts a surface of the first polymeric layer 155A along its entire area but in addition, contacts the substrate. In other words, portions of the first polymeric layer 155A can be etched away to expose a surface of the substrate 154.

If the polymer used in the first polymeric layer 155A is a copolymer, the first polymer and the second polymer may phase separate (into phase A produced by Block A and phase B produced by Block B) upon being disposed on the substrate 154. This is shown in the FIG. 4. One of the phases of the block copolymer may be etched to form the textured second surface upon which the second polymeric layer 155B is disposed. The first and the second polymeric layers may be subjected to baking if desired. Suitable baking temperatures are from 75 to 200° C., preferably 100 to 150° C.

The total thickness of the first and the second polymeric layer is about 10 to 3000 nanometers, preferably 20 to 1500 nanometers. The thickness of the layers provides the ability to manufacture small and light weight gas sensors.

When the sensing element is contacted by the fluid, certain gaseous molecules in the fluid contact the second polymeric layer and bond to it. The weight difference of the sensing element prior to and after the bonding of the gaseous molecules results in the generation of a proportional electrical current by the piezoelectric substrate. The electrical signal is calibrated to indicate to the user the molecule(s) that have interacted with the second polymeric layer 155B. The increased surface area of the free surface of the second polymeric layer 155B facilitates an increased collection of hazardous gas molecules on the surface of the sensing element, thus increasing the sensitivity of the gas sensor.

The gas sensor may thus be used to detect hazardous gases that are present in the environment in residential, commercial or industrial environments. In particular, the gas sensor is used in in refrigerators, appliances and storage areas where food products and perishable items may be stored. The gas sensor may be used to detect hazardous, unwanted or disagreeable gases such as carbon monoxide, carbon dioxide, formaldehyde, hydrogen sulfide, amines, ozone, ammonia, benzene, and so on.

Another application for the gas sensor lies in an analysis of the breath or volatile gases emitted by biological processes, or for the diagnosis of diseases. For example, human breath contains a number of volatile organic compounds (VOCs). Accurate detection of VOCs in exhaled breath can provide essential information for the early diagnosis of diseases. For example, acetone, hydrogen sulfide, ammonia, mercaptans, nitrogen monoxide and toluene can be used to evaluate diabetes, halitosis, kidney malfunction, and lung cancer, respectively, where the diagnosis of these diseases can be achieved by analyzing the concentration of VOCs in exhaled breath, originating from the molecular exchange between lung tissue and blood. Variations in the concentration of the exhaled VOCs that may serve as biomarkers for specific diseases can distinguish healthy people from those who are sick.

Another application of gas sensor detection of gases may be for the monitoring the ripening of foodstuffs such as fruits, or the over ripening of fruits, or the aging or decay of foodstuffs such as fish and meat products. For example, ripening fruit generates ethylene gas. Accurate detection of ethylene gas or other volatile gases emitted by a fruit could monitor shelf life or peak ripeness. Aging or spoiling of fish products generates amines such as trimethylamine, hydrogen sulfide, sulfur dioxide, nitrogen oxides, and ammonia, and aging or spoiling of meats generates other volatile components such as ethyl acetate, methane, carbon dioxide and ammonia. Variations in the concentration of the emitted VOCs may be used to diagnose the usefulness, quality and safety of the products.

Another application of gas sensor detection of exhaled gases may be for the monitoring the blood alcohol level of a human being for the purposes of safe operation of equipment such as cars, trucks, boats and airplanes or other industrial equipment. In addition, such gas sensor detection of exhaled gases could also have forensic or law enforcement applications. For example, variations in the concentration of alcohol, ketones and aldehydes in the breath is closely correlated with the blood alcohol level.

The gas sensor may exemplified by the following non-limiting example.

### EXAMPLE

This is a paper example to demonstrate the viability of manufacturing a gas sensor that may be used for the detection of gases. A first layer 155A comprising a block copolymer of polymethylmethacrylate and polydimethylsiloxane is disposed on a piezoelectric substrate that comprises quartz. The block copolymer is dissolved in a solvent and then disposed on the substrate. The substrate along with the block copolymer disposed thereon is annealed at a temperature of 90 C for a period of 3 hours to remove the solvent.

During the annealing, the blocks of polymethylmethacrylate phase separate from the blocks of polydimethylsiloxane to from cylinderical domains or lamellar domains. The cylindrical or lamellar domains generally comprise the polydimethylsiloxane. The block copolymer is then subjected to etching to remove the polydimethylsiloxane phase from the block copolymer leaving behind a textured second surface that can be used to detect gas molecules. The gas sensor comprising the piezoelectric substrate 154 and the textured first polymeric layer 155A is placed in contact with the appropriate electronics. The device is placed in a stream containing a trace of acetic acid. An increase in the weight of the sensor is detected by virtue of an electrical current generated by the piezoelectric substrate.

The sensor can therefore be used to detect acidic molecules that are present in the ambient atmosphere around the sensor. In one embodiment, the sensor can detect the present of undesirable or disagreeable molecules (in the atmosphere) by a weight difference of the sensor prior to and after being exposed to undesirable or disagreeable gases. In another embodiment, the sensor can detect the presence of undesirable or disagreeable molecules by means of a difference in electrical conductivity of the sensing layer 155A prior to and after being exposed to undesirable or disagreeable gases. In yet another embodiment, the sensor can detect the presence of undesirable or disagreeable molecules by virtue of a chemical analysis of molecules disposed on the sensing surface prior to and after being exposed to undesirable or disagreeable gases.

The sensor can also be provided with capabilities to replenish or refurbish the sensing surface after it has been expended in detecting various molecules of disagreeable or undesirable gases. In one embodiment, the sensor may be chemically treated to refurbish the contaminated sensor surface. In another embodiment, the sensor can be heated to a temperature effective to refurbish the contaminated surface by causing the detected gas molecules to debond from the surface. The heating to refurbish the surface can be conducted by conduction, radiation or convection.

What is claimed is:

1. A gas sensor comprising:
a piezoelectric substrate; and
a first polymeric layer disposed on the substrate; where the first polymeric layer has a first surface contacting a substrate and a second surface having a higher surface area than the first surface. where the first polymeric layer comprises a repeat unit that is effective to adsorb molecules present in the atmosphere.

2. The gas sensor of claim 1, wherein the first polymeric layer comprises a repeat unit that is operative to undergo hydrogen bonding, van der Waals interactions, π-π interactions, electrostatic interactions, or a combination thereof with an ambient gaseous molecule.

3. The gas sensor of claim 1, wherein the first polymeric layer comprises a residue of a polymer that is subjected to etching.

4. The gas sensor of claim 1, wherein the first polymeric layer comprises a residue of a block copolymer that is subjected to etching to remove at least one block of the copolymer.

5. The gas sensor of claim 1, wherein the first polymeric layer is formed from one block of a block copolymer and the second surface is in contact with the second block of the block copolymer.

6. The gas sensor of claim 1, wherein the first polymeric layer is formed from one block of a block copolymer and the second surface is exposed to the atmosphere.

7. The gas sensor of claim 5, wherein the repeat unit comprises a nitrogen-containing group or a carboxylate containing group.

8. The gas sensor of claim 7, wherein the nitrogen-containing group is selected from amine, amide and pyridine groups.

9. The gas sensor of claim 6, where the second surface is textured and where the texturing is obtained by removing a block from the first polymeric layer.

10. The gas sensor of claim 9, where the second surface has a surface area that is at least two times greater than a surface area of the first surface.

11. The gas sensor of claim 1, further comprising a surface modification layer disposed on the substrate and facilitates anchoring the first polymeric layer where the surface modification layer comprises a random copolymer that facilitates the formation of a vertically arranged morphology within an overcoated block copolymer.

12. The gas sensor of claim 5, further comprising a second polymeric layer disposed on the first polymeric layer, where a free surface of the second polymeric layer is textured and where the second polymeric layer comprises repeat units that comprise a nitrogen-containing group, a carboxylate containing group, a carboxylic acid containing group, a hydrocarbon containing group, or a combination thereof.

13. A method of manufacturing a gas sensor comprising:
disposing upon a piezoelectric substrate a first polymeric layer having a first surface and a second surface; where the second surface has a higher surface area than the first surface; and where the first polymeric layer comprises repeat units that are operative to undergo hydrogen bonding, van der Waals interactions, π-π interactions, electrostatic interactions, or a combination thereof with an ambient gaseous molecule.

14. A method of detecting a gas comprising:
contacting a gas sensor with a gaseous molecule; where the gas sensor comprises:
a piezoelectric substrate;
a first polymeric layer having a first surface and a second surface disposed on the substrate; where the second surface has a higher surface area than the first surface; and
forming at least one of a hydrogen bond, a van der Waals interaction, a π-π interaction or an electrostatic interaction between the gas molecule and the first polymeric layer; and
determining an identity of the gas molecule based on a difference of the sensor prior to and after the forming of the hydrogen bond, the van der Waals interaction, the π-π interaction and/or the electrostatic interaction.

15. The method of claim 14, where the difference is a vibration, weight difference or a difference in electrical conductivity.
